(19) United States
(12) Patent Application Publication
(10) Pub. No.: US 2009/0234300 A1
(43) Pub. Date: Sep. 17, 2009

(54) CATHETER KIT WITH A GUIDE WIRE

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/402,641

(22) Filed: Mar. 12, 2009

(30) Foreign Application Priority Data
Mar. 12, 2008 (DE) 102008013884.3

(51) Int. Cl. A61M 25/00; A61F 11/00; A61M 25/09

(52) U.S. Cl. 604/264; 606/108; 604/528

(57) ABSTRACT

A catheter kit with a guide wire (1) and a treatment catheter (3) that can be displaced to a treatment site via the guide wire (1) is additionally provided with a guide catheter (7), maneuverable at its distal end (6), having an interior lumen in order to allow acceptance the guide wire (1). The combination of the maneuverable guide catheter (7) and the guide wire (1), initially cooperating as a unit, allows the placement of said guide wire (1) even to hard to access places. After the retraction of the guide catheter (7), via the placed guide wire (1), the treatment catheter (3) can be brought into the operational position

### CROSS-REFERENCE TO RELATED APPLICATIONS

This application claims the benefit of German Application DE 10 2008 013 884.3, filed Mar. 12, 2008, which is incorporated herein by reference as if fully set forth.

### BACKGROUND

The invention relates to a catheter kit with a guide wire and with a treatment catheter that can be displaced on said guide wire to its destination, comprising an interior channel or an interior lumen, by which it encloses the guide wire in the operational position.

Such catheter kits or catheter arrangements are known in various forms and versions. In such a catheter kit the guide wire serves first to be placed such that the displacement of the treatment catheter to the respective location is facilitated for the surgeon, because the treatment catheter is only required to be displaced along the guide wire after its placement, and then can easily follow the curves and directional changes, for example inside a blood vessel, due to the appropriate arrangement of the guide wire. The relatively thin guide wire can here be placed easier than the treatment catheter, usually provided with a larger cross-section, thus such a guide wire is a component of very many catheter kits.

However, there are places inside the human body and particularly in the heart, for example at the blood outlet off the coronary veins, the so-called coronary sinus, where the addition of a guide wire is practically impossible because these places are not accessible for common guide wires.

### SUMMARY

Therefore the object is to provide a catheter arrangement or a catheter kit of the type mentioned at the outset, which allows placement of a treatment catheter and particularly its distal end in a simple fashion at a place in the human body difficult or hard to access, for example at the coronary sinus.

To attain this object, according to the invention it is provided that the catheter kit mentioned at the outset additionally includes as a component a guide catheter maneuverable at its distal end, comprising a continuous channel or interior lumen that opens at its maneuverable distal end, in which the guide wire is arranged at least during its placement and is displaceable in its operational position.

Thus, a catheter arrangement is provided, in which an additional guide catheter can be used to reach, due to its maneuverability, a point in the human body hard to access, so that the guide wire fitting into the guide catheter and at least temporarily located therein during or after the placement of the guide catheter can be pushed forward very easily and be brought into an operating position. In other words, adding said guide catheter to the catheter kit allows access to a position in the human body hard or difficult to reach, so that by the help of this controlled catheter, a guide wire can be placed using the channel located therein, which now in this guide catheter very easily and quickly accesses the hard to reach position, and then, after the controlled guide catheter has been retracted, the treatment catheter can cooperate with the guide wire in a known fashion, and thus can be inserted or implanted and/or placed via the guide wire. Here, the treatment catheter can also be an electrode with a respective interior channel or interior lumen.

Here, it is particularly beneficial when the guide catheter can be retracted and/or separated in reference to the guide wire located in its operational position, in order to release the place for inserting the treatment catheter in the above-mentioned manner.

The channel or the interior lumen of the maneuverable guide catheter may be liquid-tight. This way it is possible to use the guide catheter also to first apply a liquid, for example a drug, so that this guide catheter, being a part of the catheter kit, fulfills additional functions.

Another embodiment of the catheter kit according to the invention may provide that an expandable balloon is arranged at the guide catheter at or near its distal end. This allows, at least briefly, the ability to close the coronary sinus, thus any blood exiting a coronary vein, in order to allow during the brief interruption of the blood flow caused thereby to check, for example with the help of a contrast agent, the anatomic area in or behind the coronary sinus and to examine it, so that the subsequent placement of the guide wire and then the placement of the treatment catheter or the electrode through the coronary sinus is facilitated.

Here, inside the guide catheter a separate channel, hose, tube, or line leading to the balloon may supply a medium serving to expand the balloon.

The maneuverable guide catheter can be provided with at least one tensile element arranged eccentrically in reference to its longitudinal center, which extends to its distal end, that can be flexibly deflected, and which can be impinged with a tensile force and/or pressure to deflect the distal end of the guide catheter. Primarily when the tensile element is guided such that a transverse force can be applied the guide catheter can be deflected with its distal end to opposite sides, which facilitates the maneuverability together with the interior cross-section simultaneously being as narrow as possible, because no second tensile element is required for the deflection to opposite sides. Practically, the maneuverable guide catheter can be designed and embodied with regard to its maneuverability similar to DE 103 37 580 A1.

Here, it is beneficial for the tensile element to extend in a guide element or tube and is tightly enclosed thereby, and thus prevented from bending when transverse forces are applied.

A catheter arrangement or a catheter kit is provided, particularly from a combination of individual or several of the above-mentioned features and measures, by which even hard to reach areas inside the human body and particularly inside the heart can be well accessed, so that for the placement of the guide wire, the guide catheter being a part of the kit can be implanted temporarily due to its maneuverability with the guide wire and simultaneously or subsequently can easily be brought into its operational position through the guide catheter. Subsequently, after the removal of the guide catheter, the treatment catheter or an electrode serving for the treatment can be implanted in a conventional manner via the guide wire.

### BRIEF DESCRIPTION OF THE DRAWINGS

In the following, an exemplary embodiment of the invention is explained in greater detail using the drawing. It shows, in a partially schematic illustration:

FIG. 1 is a side view of a guide catheter being a part of a catheter kit according to the invention, which at its distal end, that can be deflected, also carries a balloon,

FIG. 2 is an view according to FIG. 1, with an injection device being connected at the inlet into the interior lumen of the guide catheter, by which a drug can be inserted through the guide catheter, with simultaneously the ability to deflect the maneuverable distal end of the guide catheter to opposite sides being shown,

FIG. 3 is a view according to FIGS. 1 and 2, in which a guide wire is inserted at another inlet to the interior lumen of the guide catheter, which projects at the distal end of the guide catheter,

FIG. 4 is a view of a guide catheter with a guide wire according to FIG. 3 after the insertion into a heart and here into the coronary sinus,

FIG. 5 is a view according to FIG. 4, in which the guide catheter is already partially retracted in reference to the guide wire initially placed with its help, and

FIG. 6 is a view according to FIGS. 4 and 5, in which it is shown that after the final retraction of the guide catheter via the guide wire a treatment catheter or an electrode, being part of the catheter kit, can be inserted with respective anchors, with the treatment electrode first being inserted partially and remaining displaceable to the end of the guide wire.

### DETAILED DESCRIPTION OF THE PREFERRED EMBODIMENTS

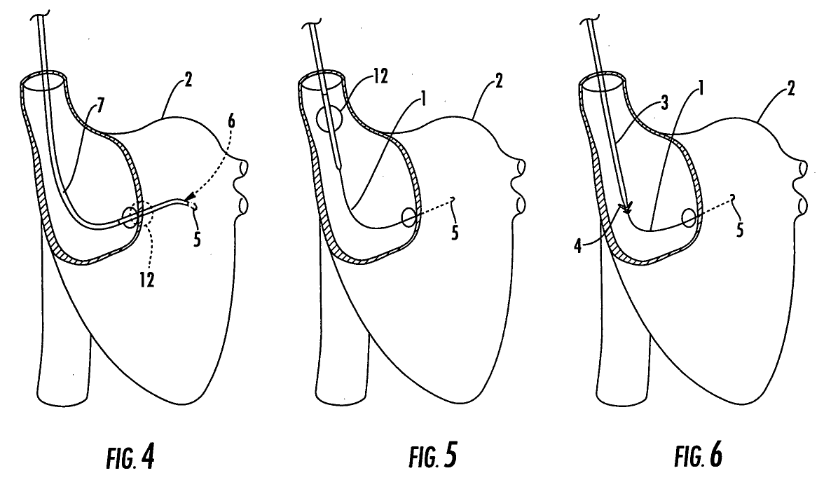

A catheter kit, not shown in its entirety in any of the figures, has as essential components a guide wire 1 and a treatment catheter 3, which can be displaced according to FIG. 6 on the guide wire 1 to its destination in a heart 2, for this purpose provided in a known manner with an interior channel or an interior lumen, by which it encloses the guide wire 1 in the operational position according to FIG. 6. In FIG. 6, an anchor 4 is discernible at the electrode and/or at the treatment catheter 3, by which the treatment catheter 3 can be fixed after the final implanting, thus after the final displacement to the end 5 of the guide wire 4.

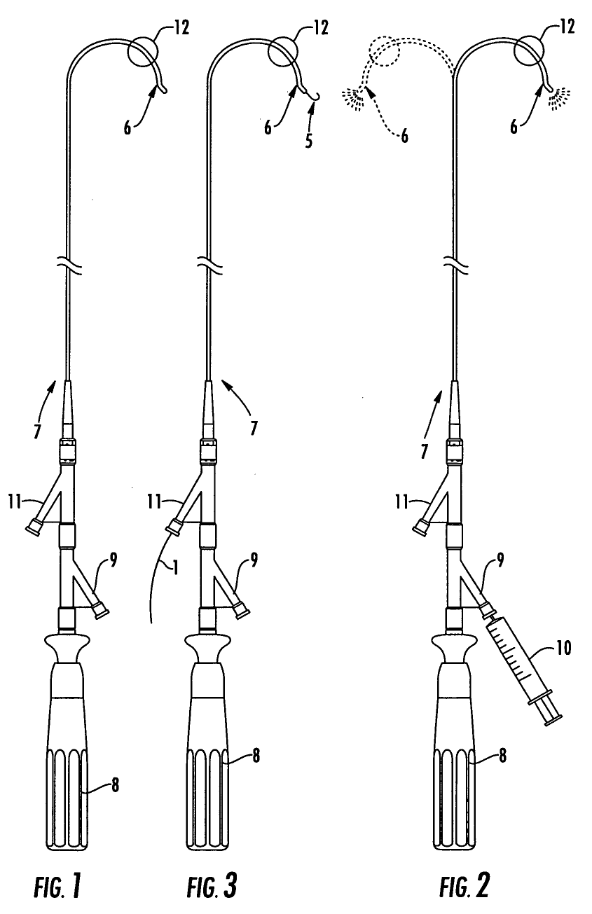

Further, this catheter kit comprises a guide catheter, clearly shown in FIGS. 1 through 3 and maneuverable at its distal end 6, in its entirety marked 7, which in the area of its handle 8, on the one hand, comprises an inlet 9 for a drug, which for example can be inserted via an injection device 10 as shown in FIG. 2, and which has another inlet 11 for the guide wire 1, which is clearly shown in FIG. 3 primarily, where the guide wire 1 has already been inserted in this guide catheter 7 and from which it slightly projects with its end 5 from the distal end 6 of the guide catheter 7. This distal end 5 of the guide wire 1 is here curved such that the guide wire 1 can engage the tissue after it has reached its operational position according to FIGS. 4 through 6.

The guide catheter 7 has therefore a continuous channel or an interior lumen, opening at its maneuverable distal end 6, in which the guide wire 1 is arranged at least during its placement, according to FIG. 4, and which is displaceable in its operational position. This way, for the placement of the guide wire 1 the maneuverability of the guide catheter 7 can be used in order to allow bringing the guide wire 1 even to difficult places, for example the coronary sinus, in a simple and quick fashion. The guide wire 1 can be brought into its operational position, together with the guide catheter 7, already inserted therein, or subsequently after the insertion of the guide catheter 7.

The guide catheter 7 can also be retracted in reference to the guide wire 1, when said wire has reached its operational position according to FIGS. 4 and 5, and in FIG. 5 this process of retracting the guide catheter 7 is illustrated. Contrary to the position shown in FIG. 4, the distal end 6 of the guide catheter 7 in FIG. 5 is already retracted a certain distance and is now close to leaving the heart 2.

The channel and the interior lumen of the maneuverable guide catheter 7 is here liquid-tight, so that, indicated in FIG. 2, it is easily possible to insert a drug through this guide catheter 7, for example into the heart 2, before the treatment catheter 3 is placed.

In FIGS. 1 through 5 it is discernible that an inflatable balloon 12 is arranged at the guide catheter 7, in the exemplary embodiment near the distal end 6. This way, perhaps according to the arrangement according to FIG. 4, the blood flow can be briefly interrupted in order to allow the examination of the area to be treated, for example via a fiber optic.

Inside the guide catheter 7 a separate channel, hose, tube, or line may be provided leading to the balloon 12, through which the medium can be guided serving to inflate the balloon 12.

Further, in a manner not described in greater detail, the maneuverable guide catheter 7 is provided with at least one tensile element, arranged eccentrically in reference to its longitudinal center and maneuverable, extending to its deflectable, bendable distal end 6 and serving to deflect the distal end 6 of the guide catheter 7 by a tensile force and/or pressure, i.e. the guide catheter 7 may be embodied with regard to its maneuverability as known from DE 103 37 580 A1, which is incorporated herein by reference as if fully set forth.

Here, the tensile element not shown in greater detail and arranged inside the guide catheter 7 can extend, in a guide element or tube and be tightly enclosed thereby, prevented from bending under pressure.

The above-described catheter arrangement, i.e. the catheter kit according to FIGS. 1 through 6, allows to place a guide wire 1 to a place inside the heart 2, which is usually not accessible by a guide wire, by the kit comprising a guide catheter 7, by which or through which a guide wire 1 can be displaced when said guide catheter 7 is placed towards the location to be accessed. Thereafter, in a common fashion, after the retraction of the guide catheter 7, the actually most important part of the catheter kit, namely the treatment catheter 3, can reach the point of treatment via the guide wire 1.

The catheter kit with a guide wire 1 and a treatment catheter 3, to be moved to the treatment area via said guide wire 1, is also provided with a guide catheter 7, maneuverable at its distal end 6, which has an interior lumen to allow accepting the guide wire 1 inside. The combination of the maneuverable guide catheter 7 and the guide wire 1, initially cooperating as a combination, allows the placement of said guide wire 1 even at areas hard to access. After the retraction of the guide catheter 7, via the placed guide wire 1, the treatment catheter 3 can be brought into its operational position.

1. A catheter kit comprising a guide wire (1), a treatment catheter (3), displaceable on the guide wire (1) to a destination, the treatment catheter including an interior channel or an interior lumen, which encloses the guide wire (1) in an operational position, a maneuverable guide catheter (7) that is maneuverable at a distal end (6) thereof, which has a continuous channel or an interior lumen opening at the maneuverable distal end (6), in which the guide wire (1) is arranged at least during placement and can be displaced into the operational position.

2. The catheter kit according to claim 1, wherein the guide catheter (7) is displaceable and retractable and can be separated from the guide wire (1).

3. The catheter kit according to claim 1, wherein the interior channel or the interior lumen of the maneuverable guide catheter (7) is liquid tight.

4. The catheter kit according to claim 1, wherein an expandable balloon (12) is arranged on the guide catheter (7) at or near the distal end (6).

5. The catheter kit according to claim 4, wherein a separate channel, hose, tube or line for a medium serving to expand the balloon (12) is arranged inside the guide catheter (7) leading to the balloon (12).

6. The catheter kit according to claim 1, wherein the maneuverable guide catheter (7) has at least one tensile element arranged eccentrically in reference to a longitudinal center thereof, which extends to the deflectable flexible distal end (6) and through which the guide catheter (7) can be impinged with a tensile force and/or pressure for deflecting the distal end (6).

7. The catheter kit according to claim 6, wherein the tensile element extends in a guide element or tube and is tightly enclosed thereby preventing bending under pressure.
